(19) United States
(12) Patent Application Publication
(10) Pub. No.: US 2022/0370058 A1
(43) Pub. Date: Nov. 24, 2022

(54) ENDOSCOPY INSTRUMENT

(71) Applicant: ProMedica Health System, Inc., Toledo, OH (US)

(72) Inventor: Tarik Z. Alhmoud, Toledo, OH (US)

(21) Appl. No.: 17/662,718

(22) Filed: May 10, 2022

Related U.S. Application Data
(60) Provisional application No. 63/186,502, filed on May 10, 2021.

(51) Int. Cl. A61B 17/00; A61B 17/221; B29C 65/48; B33Y 80/00; B29C 33/38

(52) U.S. Cl. CPC A61B 17/00234; A61B 17/221; B29C 65/4845; B33Y 80/00; B29C 33/3842; A61B 2017/00358; A61B 2017/00292; A61B 2017/00526; A61B 2017/00862; B29L 2031/753

(57) ABSTRACT

An endoscopy instrument is configured to be adjustable between a retracted position and an extended position. The endoscopy instrument includes a snare forming a loop extending from a proximate end to a distal end and a plurality of basket wires. Each of the basket wires includes a proximate end coupled to the proximate end of the snare and a distal end coupled to the distal end of the snare. The snare and the plurality of the basket wires cooperate to form a basket portion configured to contract when the endoscopy instrument is adjusted from the extended position towards the retracted position during entry of the basket portion into a tubular member. The contracting of the basket portion aids in grasping an object in need of retrieval from a patient.

### CROSS-REFERENCE TO RELATED APPLICATION

This patent application claims priority to U.S. Provisional Patent Application Ser. No. 63/186,502, filed on May 10, 2021, the entire disclosure of which is hereby incorporated herein by reference.

### FIELD OF THE INVENTION

The invention relates to an endoscopy instrument, and more particularly, to a multi-feature endoscopy instrument for removing objects from a patient, such as foreign bodies or tissue formations present within the patient.

### BACKGROUND OF THE INVENTION

It is common for food impactions or other foreign bodies to be lodged within the esophagus, stomach, duodenum, rectum, or colon of a patient in a manner requiring medical intervention. In the instance of food impactions, the food items can range from large pieces of meat or chicken to softer foods such as noodles or vegetables. The various different types of foods or other foreign bodies, as well as the unique shapes and configurations thereof, may lead to the need for multiple different tools to be utilized for performing a single retrieval procedure. As a result, such retrieval procedures can take as long as several hours if difficulties are encountered, wherein many of the difficulties result from the lack of sufficient tools for retrieving the given food impaction or foreign body. The prolonging of such procedures may increase the risk to the patient while also undesirably consuming valuable time of the medical practitioner needed to address other patients similarly requiring care.

Such tools are also not limited in use to the removal of ingested foreign bodies, and may alternatively be utilized to remove tissue from within the body of the patient as required by certain procedures. As one example, severe pancreatitis can lead to necrosis and the formation of fluid collections or walled-off necrosis in the pancreas. Complicated fluid collections in the pancreas may require drainage by surgery, interventional radiology (percutaneous drainage), or endoscopic drainage. When endoscopic access is required, such access is established from the stomach into the pancreatic cyst using Lumen-apposing metal stents (LAMS). Various upper endoscopic instruments may be passed through the stent into the pancreatic cystic cavity to allow the encountered necrotic tissue to be fragmented and partially removed, wherein the removal of such necrotic tissue may require multiple passes of the various different instruments. As another example, large polyps may be found in the colon, stomach, or small bowel during screening colonoscopy or upper endoscopy procedures, wherein such polyps must be removed and retrieved from within the body of the patient utilizing various different endoscopic tools.

The endoscopy tools of the prior art tend to have structures and configurations that cause such tools to be undesirably limited in application, thereby prolonging and complicating the removal of the food impaction, foreign body, or tissue sample, depending on the procedure. Examples of such insufficient endoscopy tools or instruments include grasping tools, retrieval baskets, nets, forceps, and snares, each of which has its own shortcomings and inefficiencies. The grasping tools tend to have limited maneuverability and often do not open wide enough to grasp the given object. The retrieval baskets are not well suited for removing soft or large food impactions. The nets tend to break easily following multiple uses thereof and also fail to easily locate and grasp the foreign object within the net. The forceps tend to only be able to grasp relatively small objects and hence are not applicable to variable applications. Finally, the snares have difficulty in grasping and maintaining the grasp on the foreign objects while also presenting a risk of cutting through the foreign object during the grasping process. These shortcomings make it difficult to utilize any of the mentioned tools for more than a limited set of circumstances that may not end up applying to the instantaneous procedure.

It would accordingly be desirable to produce an endoscopy instrument that is versatile and capable of being utilized for grasping and retrieving a variety of different foreign bodies, food impactions, or tissue samples.

### SUMMARY OF THE INVENTION

Consonant with the present disclosure, a new and improved endoscopy instrument has surprisingly been discovered.

According to an embodiment of the present invention, an endoscopy instrument is configured to be adjustable between a retracted position and an extended position. The endoscopy instrument includes a snare forming a loop extending from a proximate end to a distal end and a plurality of basket wires. Each of the basket wires includes a proximate end coupled to the proximate end of the snare and a distal end coupled to the distal end of the snare. The snare and the plurality of the basket wires cooperate to form a basket portion configured to contract when the endoscopy instrument is adjusted from the extended position towards the retracted position during entry of the basket portion into a tubular member. The contracting of the basket portion aids in grasping an object in need of retrieval from a patient.

A method of manufacturing an endoscopy instrument is also disclosed. The method includes the steps of: providing a barb-forming mold having a channel and a barb recess formed therein, the barb recess extending laterally from the channel; positioning an elongate element of the endoscopy instrument within the channel of the barb-forming mold; inserting a curable material into the barb recess; and curing the curable material to form a barb coupled to the elongate element.

### BRIEF DESCRIPTION OF THE DRAWINGS

Further particulars, features and advantages of the embodiments of the invention result from the subsequent description of embodiments with reference to the associated drawings. The drawings show:

FIG. 1 is a side elevational view of an endoscopy instrument according to an embodiment of the present invention, wherein the endoscopy instrument is shown installed within an exemplary device configured to adjust a configuration of the endoscopy instrument;

FIG. 2 is front elevational view of the endoscopy instrument of FIG. 1;

FIG. 3 is a right elevational view of the endoscopy instrument of FIG. 1;

FIG. 4 is a top plan view of the endoscopy instrument of FIG. 1;

FIG. 5 is a perspective view showing a first method of use of the endoscopy instrument relative to an object in need of removal;

FIG. 6 is a perspective view showing a second method of use of the endoscopy instrument relative to an object in need of removal;

FIG. 7 is a perspective view showing a third method of use of the endoscopy instrument relative to an object in need of removal;

FIG. 8 is a perspective view of an endoscopy instrument having a plurality of barbs extending therefrom;

FIG. 9 is an enlarged view of a portion of the endoscopy instrument having the barbs as shown within the circle 9 of FIG. 8.

FIG. 10 is a perspective view of an endoscopy instrument having a net coupled thereto;

FIG. 11 is a perspective view of a first mold utilized in manufacturing a barb for projection from a basket wire of the endoscopy instrument;

FIG. 12 is a perspective view showing the formation of a second mold created via a molding process performed with respect to the first mold;

FIG. 13 is a perspective view of the second mold of FIG. 12 following formation thereof;

FIG. 14 is a perspective view of the second mold with a basket wire received within a channel formed within the second mold;

FIG. 15 is an enlarged view of a portion of FIG. 14 shown within circle 15; and

FIG. 16 is a perspective view of a barb extending from a basket wire as produced by the method disclosed in FIGS. 11-15.

### DETAILED DESCRIPTION OF EXEMPLARY EMBODIMENTS OF THE INVENTION

The following detailed description and appended drawings describe and illustrate various exemplary embodiments of the invention. The description and drawings serve to enable one skilled in the art to make and use the invention, and are not intended to limit the scope of the invention in any manner.

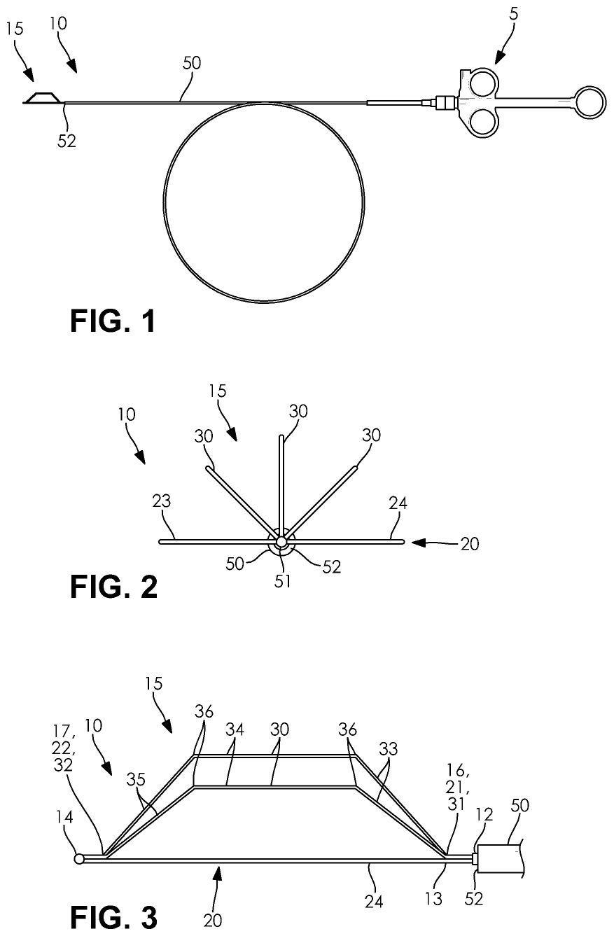

FIGS. 1-4 illustrate an endoscopy instrument 10 according to an embodiment of the present invention. The endoscopy instrument 10 may be associated with the retrieval of various different foreign bodies, food impactions lodged within the patient, or tissue portions or samples removed from the patient. As used hereinafter, all such bodies capable of being retrieved by the endoscopy instrument 10 are generally referred to as the “object” being removed for simplicity and brevity. Such objects may be removed from any portion, region, or opening of the body of the patient suitable for use with the endoscopy instrument 10 as described hereinafter. As described in the background of the present invention, such regions or portions of the body may include the esophagus, stomach, duodenum, rectum, colon, or pancreatic cystic cavity, as non-limiting examples. It should be apparent to one skilled in the art that the endoscopy instrument 10 may be adapted for use with substantially any cavity or opening within the body of the patient and may be utilized in removing substantially any solid object therefrom while remaining within the scope of the present invention, as desired.

The endoscopy instrument 10 may be adapted for use with any endoscope, catheter, stent, or similar device suitable for locating the endoscopy instrument 10 adjacent the associated object within the body of the patient. The endoscopy instrument 10 may also be associated with any type of device having an ability to visualize the interior of the cavity or opening of the patient during use of the endoscopy instrument 10 as described hereinafter. The associated device may accordingly include multiple different lumens or other tubular structures associated with placing the endoscopy instrument 10 at the desired position and configuration for retrieving the object as described hereinafter without departing from the scope of the present invention. Furthermore, the endoscopy instrument 10 may be associated with any type of actuating mechanism of the associated device for extending and retracting the endoscopy instrument 10 and any other associated components (such as the associated visualization component) during use thereof without departing from the scope of the present invention. Lastly, the associated device may include any type of handle or control mechanism for controlling the movements of the tubular structure retractably housing the endoscopy instrument 10 without departing from the scope of the present invention. The endoscopy instrument 10 is shown in FIG. 1 as being utilized in conjunction with a device 5 configured to operate the endoscopy instrument 10 in accordance with the description of the endoscopy instrument 10 as set forth hereinafter.

The endoscopy instrument 10 generally includes a stem 12 and a basket portion 15 comprising a snare 20 and a plurality of basket wires 30. The endoscopy instrument 10 is also configured for extension from and retraction into a tubular member 50. The tubular member 50 guides the stem 12 and includes a length sufficient for locating the endoscopy instrument 10 relative to the object in need of removal via operation of the corresponding device 5.

The tubular member 50 may define a hollow passageway 51 having an inner diameter suitable for housing the endoscopy instrument 10 when the endoscopy instrument 10 is in a retracted or collapsed position within the tubular member 50. In turn, the inner diameter of the hollow passageway 51 formed through the tubular member 50 is also smaller than a corresponding dimension of the basket portion 15 of the endoscopy instrument 10 when the basket portion 15 is in the extended or opened position thereof. As explained hereinafter, the extension of the basket portion 15 comprising the snare 20 and the basket wires 30 out of the tubular member 50 facilitates the progressive opening of the basket portion 15 to the open position thereof while the retraction of the basket portion 15 back into the tubular member 50 facilitates the progressive collapsing of the basket portion 15 to the collapsed position thereof. The tubular member 50 may be, for example, a lumen associated with the device used to position the endoscopy instrument 10 adjacent the object in need of retrieval. The tubular member 50 may itself be extendable or retractable relative to a corresponding endoscope, catheter, or the like forming the device 5 for placing an end 52 of the tubular member 50 from which the endoscopy instrument 10 extends adjacent the object in need of removal. However, it should be readily apparent to one skilled in the art that substantially any mechanism causing an end 13 of the stem 12 of the endoscopy instrument 10 to move relative to the end 52 of the tubular member 50 for selectively extending the basket portion 15 into or out of the passageway 51 of the tubular member 50 may be utilized without departing from the scope of the present invention.

The endoscopy instrument 10 is shown in FIGS. 1-4 when the basket portion 15 is in the fully extended and opened position thereof to better illustrate and describe the relevant features thereof. Further references to any one of the snare 20 or the basket wires 30 being in the open or extended position thereof accordingly corresponds to the open or extended position of the entirety of the basket portion 15 more generally. Similarly, any one of the snare 20 or the basket wires 30 being described as being in the collapsed or retracted position thereof corresponds to the entirety of the basket portion 15 being in the collapsed or retracted position thereof

The stem 12 is formed by an elongate member fed through the tubular member 50 with the basket portion 15 comprising the snare 20 and the basket wires 30 extending from a distal end 13 of the stem 12. The stem 12 may be formed by any elongate member such as a wire, a tube, or the like. The stem 12 may be formed from a flexible material capable of bending to the desired configuration when locating the endoscopy instrument 10 adjacent the object in need of removal. The general direction of extension of the stem 12 through the tubular member 50 is hereinafter referred to as the longitudinal direction of the endoscopy instrument 10, which also coincides with the described longitudinal directions of the basket portion 15, the snare 20, and the basket wires 30 as used hereinafter. The longitudinal direction of the endoscopy instrument 10 also corresponds to the longitudinal direction of the tubular member 50 at the end 52 of the tubular member 50 from which the endoscopy instrument 10 is selectively extended/retracted.

The basket portion 15 extends longitudinally from a proximate end 16 to an opposing distal end 17. The proximate end 16 of the basket portion 15 coincides with the end 13 of the stem 12 while the distal end 17 is spaced apart longitudinally from the end 13 of the stem 12. The proximate end 16 and the distal end 17 are spaced apart from each other with respect to a central axis of the stem 12 at the end 13 thereof. In other words, the proximate end 16 and the distal end 17 of the basket portion 15 are each disposed on an axis extending from the end 13 of the stem 12. In the illustrated embodiment, the distal end 17 of the basket portion 15 includes a hub structure 14 for connection to the snare 20 and the basket wires 30, as explained in greater detail hereinafter. However, the basket portion 15 may be provided in the absence of an independently provided hub structure 14 while remaining within the scope of the present invention.

The snare 20 is formed into a loop shape capable of extending around the object in need of removal when placed in the opened position thereof. The snare 20 extends longitudinally from a proximate end 21 to a distal end 22, wherein the proximate end 21 of the snare 20 coincides with the proximate end 16 of the basket portion 15 and the distal end 22 of the snare 20 coincides with the distal end 17 of the basket portion 15. The proximate and distal ends 21, 22 are accordingly spaced apart along the central axis extending from the end 13 of the stem 12.

The snare 20 is comprised of a pair of snare segments 23, 24, wherein the snare segments 23, 24 are arranged substantially co-planar to each other. The central axis of the stem 12 at the end 13 thereof is arranged on, and is hence parallel to, the common plane occupied by the snare segments 23, 24. The snare segments 23, 24 are also arranged symmetrically relative to one another with respect to opposing sides of the central axis of the stem 12. Each of the snare segments 23, 24 extends arcuately from the proximate end 21 of the snare 20 to the distal end 22 thereof

In the illustrated embodiment, each of the snare segments 23, 24 is securely coupled to the hub structure 14 disposed at the distal end 22 of the snare 20. In other embodiments, the snare segments 23, 24 are continuous with each other at the distal end 22 for forming a looped shape. In other embodiments, the snare segments 23, 24 may be mechanically joined to one another, or otherwise aggressively joined to one another. In any event, the snare segments 23, 24 meet at the distal end 22 of the snare 20, which is configured to remain in substantial alignment with the end 13 of the stem 12 regardless of the configuration of the basket portion 15.

In the illustrated embodiment, each of the snare segments 23, 24 is provided as an outwardly projecting extension of a portion of the end 13 of the stem 12 at the proximate end 21 of the snare 20, thereby establishing a secure coupling of each of the snare segments 23, 24 to the end 13 of the stem 12 via an integral or monolithic formation therewith. In other embodiments, the snare segments 23, 24 may be otherwise coupled to the end 13 of the stem 12, such as by mechanical coupling or the use of an aggressive joining process. In any event, the snare segments 23, 24 remain affixed to the end 13 of the stem 12 at the proximate end 21 of the snare 20 regardless of the configuration of the basket portion 15 when moved relative to the end 52 of the tubular member 50.

The snare 20, as formed by the cooperation of the snare segments 23, 24, may have a substantially circular, elliptical, oval-shaped, or rounded diamond-shaped perimeter shape when in the open/extended position. In the provided embodiment, the snare 20 is shown as having a substantially elliptical perimeter shape with the elliptical shape elongated in the longitudinal direction of the snare 20, thereby resulting in each of the snare segments 23, 24 having symmetrically arranged semi-elliptical shapes. Each of the snare segments 23, 24 forming the snare 20 may be provided with a thickness sufficient for preventing the interaction/engagement occurring between the object and the snare 20 from undesirably deforming the snare 20 during the process of grasping and/or retrieving the object in need of removal.

The illustrated example of the endoscopy instrument 10 includes three of the basket wires 30 with each of the basket wires 30 including a proximate end 31 and a distal end 32. The proximate end 31 of each of the basket wires 30 is disposed to substantially coincide with the proximate end 21 of the snare 20 and the proximate end 16 of the basket portion 15 more generally, whereas the distal end 32 of each of the basket wires 30 is disposed to substantially coincide with the distal end 22 of the snare 20 and the distal end 17 of the basket portion 15 more generally.

In the illustrated embodiment, each of the basket wires 30 is securely coupled to the hub structure 14 disposed at the distal end 32 of the corresponding basket wire 30. In other embodiments, the basket wires 30 are mechanically joined to one another, or otherwise aggressively joined to one another, at the distal ends 32 thereof in the absence of the hub structure 14. In either circumstance, the distal end 32 of each of the basket wires 30 may be considered to be coupled to the snare 20 at the distal end 22 thereof, whether indirectly via the common coupling to the hub structure 14 or directly via a mechanical or aggressively joined connection present between the distal ends 32 of the basket wires 30 and the distal end 22 of the snare 20 at the distal end 17 of the basket portion 15. In any event, the distal ends 32 of the basket wires 30 meet each other at the distal end 22 of the snare 20 in a manner wherein the distal ends 32 remain in substantial alignment with the end 13 of the stem 12 regardless of the configuration of the basket portion 15.

In the illustrated embodiment, each of the basket wires 30 is provided as an outwardly projecting extension of a portion of the end 13 of the stem 12 at the proximate end 31 of the corresponding basket wire 30, thereby establishing a secure coupling of each of the basket wires 30 to the end 13 of the stem 12 via an integral or monolithic formation therewith. In other embodiments, the basket wires 30 may be otherwise coupled to the end 13 of the stem 12, such as by mechanical coupling or the use of an aggressive joining process. In any event, the basket wires 30 remain affixed to the end 13 of the stem 12 at the proximate end 31 of each respective basket wire 30 regardless of the configuration of the basket portion 15 when moved relative to the end 52 of the tubular member 50. In some embodiments, each of the basket wires 30 may be formed to be relatively thinner than the snare 20 is about the periphery thereof to render the basket wires 30 as more flexible and compliant than the snare 20, as desired. However, in other embodiments, the basket wires 30 and the snare 20 may have the same thicknesses/diameters, as desired.

Each of the basket wires 30 is shown as including a proximate segment 33, a central segment 34, and a distal segment 35, each of which is separated from one another by a bend 36 formed in the corresponding basket wire 30 where an inclination of the corresponding basket wire 30 changes relative to the central axis of the stem 12 at the end 13 thereof. The proximate segment 33 extends rectilinearly from the proximate end 31 of the corresponding basket wire 30 to a first one of the bends 36 separating the proximate segment 33 from the central segment 34. The proximate segment 33 of each of the basket wires 30 is inclined to extend at least partially radially outwardly relative to the central axis of the stem 12 at the end 13 thereof when progressing in a direction from the proximate end 16 to the distal end 17 of the basket portion 15. The central segment 34 of each of the basket wires 30 is radially spaced apart from the central axis of the stem 12 at the end 13 thereof. The central segment 34 of each of the basket wires 30 is further arranged to extend rectilinearly in parallel to the central axis of the stem 12 at the end 13 thereof. The distal segment 35 extends rectilinearly from a second one of the bends 36 separating the central segment 34 from the distal segment 35 to the distal end 32 of the corresponding basket wire 30. The distal segment 35 of each of the basket wires 30 is inclined to extend at least partially radially inwardly relative to the central axis of the stem 12 at the end 13 thereof when progressing in the direction from the proximate end 16 to the distal end 17 of the basket portion 15. The proximate segment 33 and the distal segment 35 may be inclined at the same acute angle relative to the central axis of the stem 12 at the end 13 thereof with the angles of inclination opposing one another. The described configuration of the segments 33, 34, 35 may result in each of the basket wires 30 cooperating with the central axis of the stem 12 at the end 13 thereof to form a substantially trapezoidal shape, wherein such a trapezoidal shape may be further described as being acute or isosceles in configuration.

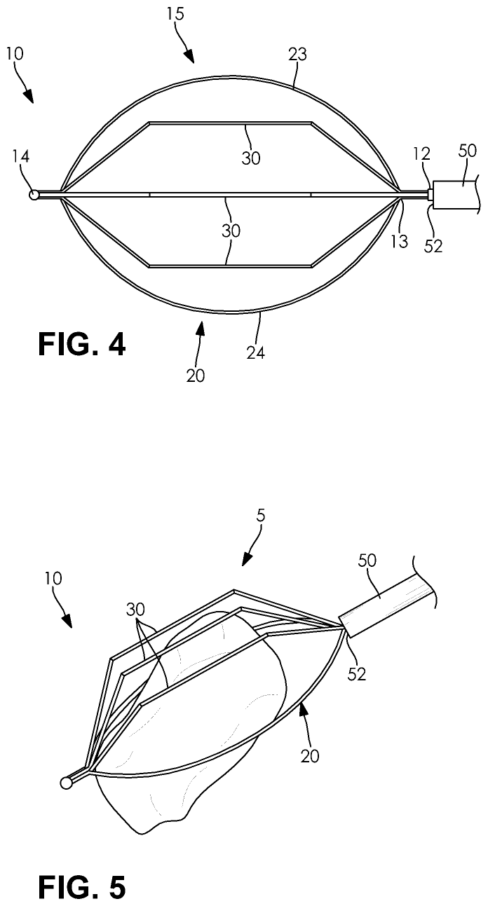

As can be seen in FIGS. 2 and 4, the plurality of the basket wires 30 includes a centrally disposed one of the basket wires 30 and a pair of laterally disposed basket wires 30 with each of the laterally disposed basket wires 30 straddling the centrally disposed one of the basket wires 30. The centrally disposed one of the basket wires 30 includes the central segment 34 thereof spaced further from a plane defined by the periphery of the snare 20 than is the central segment 34 of each of the pair of the laterally disposed basket wires 30. The central segment 34 of each of the laterally disposed basket wires 30 is also spaced laterally outwardly from a plane arranged perpendicular to the periphery of the snare 20 and extending through the centrally disposed one of the basket wires 30.

As best shown in FIG. 2, each of the snare segments 23, 24 and each of the individual basket wires 30 is disposed to extend along a corresponding plane extending radially outwardly from the central axis of the stem 12 at the end 12 thereof. As mentioned previously, the snare segments 23, 24 are formed to opposing sides of the central axis and are arranged coplanar to each other. Each of the laterally disposed basket wires 30 is disposed on a plane that is offset by about 45° from an adjacent one of the snare segments 23, 24 while the centrally disposed basket wire 30 is disposed on a plane that is offset by about 45° from each of the straddling laterally disposed basket wires 30. The illustrated embodiment accordingly includes the snare segments 23, 24 and the basket wires 30 having an equal angular spacing therebetween relative to the central axis of the stem 12. However, it is within the scope of the present invention for the angular spacing to differ between any adjacent set of the radially extending elements 23, 24, 30, as desired.

The illustrated configuration of the snare segments 23, 24 and the basket wires 30 results in the basket portion 15 being substantially dome shaped in configuration. More specifically, the snare segments 23, 24 themselves and the bends 36 separating each of the segments 33, 34, 35 of each of the basket wires 30 may each be disposed to be arranged on a common dome shape, which may be symmetric about one or more planes. Specifically, the dome shape may be symmetric about a pair of perpendicular arranged planes. If the snare 20 is substantially circular in shape, the snare segments 23, 24 and the bends 36 may be disposed for arrangement along a substantially hemispherical dome shape. Alternatively, if the basket portion 15 is elongated as illustrated in FIGS. 1-4, the snare segments 23, 24 and the bends 36 may be disposed for arrangement along a substantially hemi-ellipsoidal dome shape or a substantially semi-ellipsoidal dome shape. However, the basket portion 15 may form alternative cup or container shapes, including shapes that are not symmetrical about at least two axes, while remaining within the scope of the present invention.

Although the basket wires 30 are shown as having the trapezoidal shape including a pair of the bends 36 formed in each of the basket wires 30 for forming three rectilinear segments 33, 34, 35, it should be apparent that the basket wires 30 may be formed from any number of rectilinear segments separated from each other by any number of corresponding bends 36 for forming the same general shape of the basket portion 15 as shown and described, including the formation of any of the described dome shapes. As the number of the bends and the corresponding number of the linear segments is increased, each of the basket wires 30 may take on a more arcuate shape when extending from the proximate end 31 to the distal end 32 thereof. Furthermore, it should accordingly be apparent that each of the basket wires 30 may alternatively be formed in the absence of the bends and the linear segments to be provided as having a substantially arcuate and curved shape when extending from the proximate end 31 to the distal end 32 thereof while still taking on the same general configuration as shown and described with reference to the basket wires 30 having the bends and linear segments.

The basket portion 15 is also shown as including only three of the basket wires 30, but the basket portion 15 may be provided with any number of the basket wires 30 while maintaining the same general configurations described herein without necessarily departing from the scope of the present invention. Such a modified basket portion 15 may still include the disclosed angular spacing present between adjacent radially extending elements 23, 24, 30 for maintaining the dome shape of the corresponding basket portion 15. Such angular spacing may be selected to be equal between adjacent elements 23, 24, 30 or may be variable between the different elements 23, 24, 30, as desired.

The snare 20 and each of the basket wires 30 may be formed from the same material. The material may be a resiliently flexible material to allow for the snare 20 and each of the basket wires 30 to be reliably formed into the configuration shown in FIGS. 1-4 when placed in the fully extended and opened position of the endoscopy instrument 10 following placement of the endoscopy instrument 10 in the retracted and collapsed position thereof within the tubular member 50. The material may preferably be a metallic material having the desired strength and resiliency, but other materials, such as suitable polymeric materials, may also be utilized without necessarily departing from the scope of the present invention. According to one embodiment, each of the snare segments 23, 24 and each of the basket wires 30 is formed from a length of twisted stainless steel spring wire. The stem 12 may also be formed from the same material as the snare 20 and the basket wires 30, as desired. However, each of the stem 12, the snare 20, and the basket wires 30 may be formed from different materials, so long as each of the materials utilized maintains the properties described herein.

The tubular member 50 may be formed from a flexible and lubricious material allowing for an ease of sliding of the endoscopy instrument 10 therein. The material forming the tubular member 50 may be a polymeric material such as PTFE, FEP, or HDPE, as non-limiting examples.

The endoscopy instrument 10 as illustrated in FIGS. 1-4 may include the following relationships regarding the size and configuration of each of the described features thereof, although it should be readily apparent to one skilled in the art that such features may be modified in accordance with the examples provided herein. The embodiment shown in FIG. 1 may include the stem 12 and/or the tubular member 50 extending about 240 cm from the end of the device 5 to allow the basket portion 15 to reach the object within the patient. An outer diameter of the tubular member may be selected to be about 2.8 mm to facilitate use with a corresponding extension/retraction device such as the illustrated device 5. A lateral width of the snare 20 when in the open configuration, as measured between distal surfaces of the opposing snare segments 23, 24 in a direction perpendicular to the longitudinal direction of the basket portion 15, may be within a range of about 11-30 mm, wherein the illustrated embodiment includes a width of about 25 mm. A height of the basket portion 15, as measured from the plane defined by the snare 20 to a furthest one of the central segments 34 of the basket wires 30, may be selected to be about 40-50% of the width of the snare 20. In the illustrated embodiment, the height is 44% of the width, which corresponds to 11 mm. Lastly, the length of the basket portion 15 may be selected to be about 150% of the width thereof, which corresponds to about a 38 mm length in the illustrated embodiment. However, these dimensions are non-limiting, as alternative shapes having the same general configuration to those shown and described herein may be utilized without necessarily departing from the scope of the present invention.

As mentioned above, the endoscopy instrument 10 is shown and described when in the extended and opened position with the outermost surfaces of the snare 20 and the basket wires 30 disposed a maximum distance from the central axis of the stem 12 at the end 13 thereof. The snare 20 and each of the basket wires 30 are configured to collapse inwardly as they are progressively retracted into the passageway 51 formed through the tubular member 50. The opposing lateral sides of the snare 20 extending between the proximate end 21 and the distal end 22 thereof are brought adjacent each other while the segments 33, 34, 35 forming each of the basket wires 30 are brought into substantial longitudinal alignment with each other to allow for the snare 20 and the basket wires 30 to be received within the hollow passageway 51 of the tubular member 50. The collapsing of the snare 20 and each of the basket wires 30 in a direction perpendicular to the longitudinal direction of the stem 12 accordingly causes the snare 20 and each of the basket wires 30 to extend primarily in the longitudinal direction of the stem 12 as the components are narrowed for fitting into the hollow passageway 51 of the tubular member 50. Once fully received within the tubular member 50, the endoscopy instrument 10 is placed in the fully retracted and collapsed position.

As explained hereinabove, the endoscopy instrument 10 may be adapted for use with a variety of different tools for application during a variety of different procedures associated with a variety of different openings or cavities within a patient. The different uses of the endoscopy instrument 10 as described with reference to FIGS. 5-7 may be applied to any such procedures, where applicable, following placement of the end 52 of the tubular member 50 adjacent the object in need of removal of the corresponding opening or cavity.

Referring to FIG. 5, one use of the endoscopy instrument 10 may include grasping the object within the basket portion 15 prior to the removal of the object from the corresponding opening or cavity within the patient. Specifically, the basket portion 15 is first extended from the end 52 of the tubular member 50 to via a pushing of the stem 12 in the longitudinal direction thereof. The resiliency of the material forming the endoscopy instrument 10 causes the snare 20 and the basket wires 30 to spring to the fully extended and opened positions. The object is then placed within the looped shape of the snare 20 while also placing the object at least partially within the dome shape formed by the entirety of the basket portion 15. The snare 20 and the basket wires 30 may then be partially retracted back into the tubular member 50 via a pulling on the stem 12 in the longitudinal direction thereof to cause a partial collapsing of the endoscopy instrument 10 for more tightly grasping the object with the snare 20 and/or the basket wires 30. The retraction of the endoscopy instrument 10 results in the inner facing surfaces of the snare 20 and each of the basket wires 30 constricting towards the object, thereby constricting the object from moving in multiple direction via engagement with multiple of the radially extending elements 23, 24, 30. The tubular member 50 and the endoscopy instrument 10 can then be removed from the opening or cavity of the patient with the object grasped by the endoscopy instrument 10.

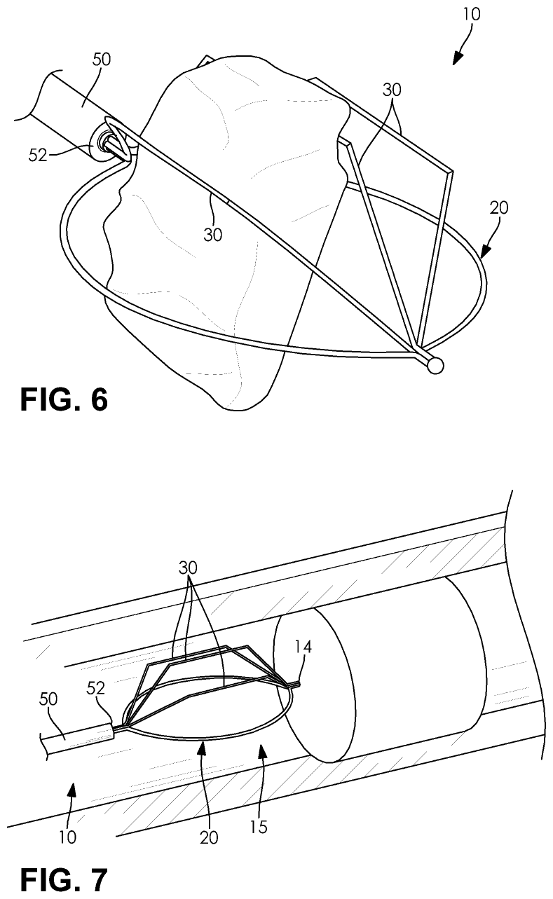

Referring now to FIG. 6, another related use of the endoscopy instrument 10 corresponds to the manner in which gaps are formed between the adjacent ones of the radially extending elements 23, 24, 30 such that the object may be grasped between such adjacent elements 23, 24, 30 during the process of constricting the basket portion 15 to the closed position. For example, as shown in FIG. 6, the object may be disposed to extend through the gap present between one of the laterally disposed basket wires 30 and the centrally disposed basket wire 30. The object may accordingly be grasped via a closing of the gap between the adjacent basket wires 30 to grasp the object even when the object is otherwise not well positioned for being collapsed upon by the remainder of the basket portion 15.

FIG. 7 illustrates the manner in which the endoscopy instrument 10 may also be fed through the corresponding opening or cavity for abutting the object at the distal end 17 of the basket portion 14, which may correspond to the hub structure 14. The distal end 17 of the basket portion 15 can be used to push the object, to partially penetrate the object, or to fully penetrate the object. The basket portion 15 may also be adjusted relative to the end 52 of the tubular member 50 for adjusting the stiffness of the basket portion 15 with respect to the longitudinal direction thereof when the basket portion 15 is narrowed and lengthened during retraction into the passageway 51.

The disclosed endoscopy instrument 10 is accordingly versatile in the ability of the endoscopy instrument 10 to interact with an object in a variety of different ways. Such versatility beneficially allows the operator of the endoscopy instrument 10 to adjust to the conditions faced during a procedure utilizing the endoscopy instrument 10 when initial or preferred strategies are unsuccessful.

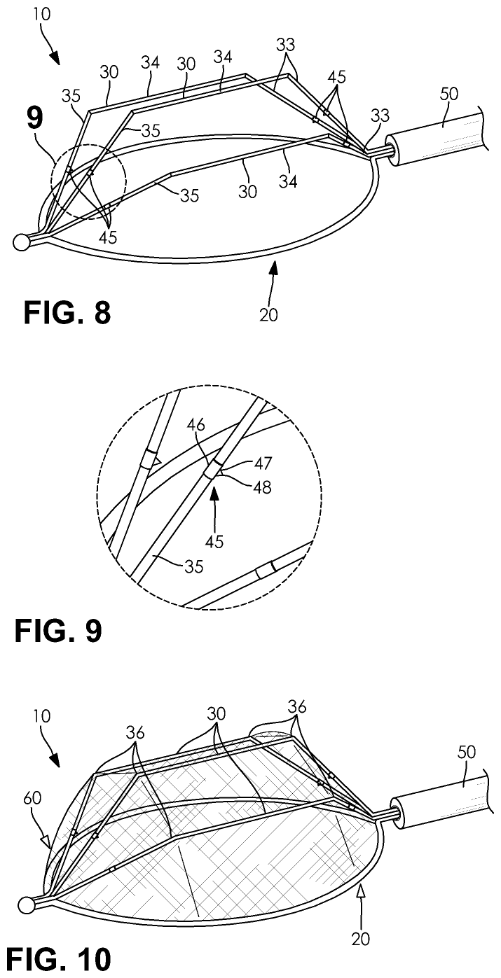

Referring now to FIGS. 8 and 9, the endoscopy instrument 10 is shown as including an additional feature wherein each of the basket wires 30 further includes a pair of teeth, hooks, or barbs 45 extending therefrom with each individual barb 45 projecting in a direction towards an interior of the basket portion 15 (towards the plane formed by the periphery of the snare 20). In the illustrated embodiment, the barbs 45 are shown as being formed within each of the proximate segment 33 and the distal segment 35 of each of the basket wires 30, but the barbs 45 may also be formed within the central segment 34 without necessarily departing from the scope of the present invention. Each of the segments 33, 35 is shown as including a single barb 45, but the segments 33, 35 may include any number of the barbs 45 at any spacing intervals therebetween while remaining within the scope of the present invention. The barbs 45 are configured to catch on the object in need of retrieval to prevent an undesired removal of the object from the basket portion 15.

As shown in the enlarged view of FIG. 9, each barb 45 may comprise a collar portion 46 extending at least partially around the corresponding segment 33, 34, 35 of one of the basket wires 30 as well as a projecting portion 47 projecting away from an inner facing surface of the collar portion 46. The projecting portion 47 of each of the barbs 45 is shown in FIGS. 8 and 9 as having a substantially triangular cross-sectional shape. However, as shown and described hereinafter when discussing one method of manufacturing the barbs 45, the projecting portion 47 of each of the barbs 45 may include a curvature towards one of the ends 16, 17 of the basket portion, or alternatively towards one of the lateral directions perpendicular to the longitudinal direction of the basket portion 15, while remaining within the scope of the present invention. Such a barb 45 may include a projecting portion 47 having at least one arcuate or curved surface, including at least one surface that is concave and/or convex. The barb 45 may include a projecting portion 47 having a concave surface and an opposing convex surface intersecting the concave surface. The barb 45 may alternatively include a projecting portion 47 having a planar surface intersecting the concave or convex surface. The described surfaces may meet at a pointed end 48 of the projecting portion 47 configured to point towards the interior of the basket portion 15, which (typically) corresponds to the direction towards the object in need of grasping.

Referring now to FIG. 10, the endoscopy instrument 10 is shown as including another optional feature in the form of a net 60. The net 60 may be coupled to the snare 20 about the periphery thereof with the net 60 disposed to extend outside of the basket portion 15 as formed by the plurality of the basket wires 30. The net 60 may form substantially the same general shape as the basket portion 15 when the endoscopy instrument 10 is placed in the fully opened position. The net 60 may additionally be coupled to each of the basket wires 30, such as at the bend 36 of each of the basket wires 30, as desired, to aid in forming the net 60 to the desired configuration when the endoscopy instrument 10 is placed in the fully opened position. The net 60 is formed from any flexible material capable of maintaining a coupling of the net 60 to the snare 20 when the endoscopy instrument 10 is adjusted between the collapsed and opened positions. The net 60 may be provided as any pattern of grid-like threads or wires forming smaller openings than the openings present between adjacent ones of the basket wires 30.

The net 60 is configured to provide an additional catching feature for catching any portions of the object that may be break off during the retrieval of the object from the patient. For example, if the object is a piece of food, the net 60 may catch any small food particles that fall away from the piece of food during the process of collapsing the snare 20 and the basket wires 30 around the piece of food. The net 60 accordingly provides an additional safety feature for preventing the object or any portion thereof from falling through the basket portion 15 in a manner that could cause the object or portion thereof to fall back into the opening or cavity of the patient.

The disclosed endoscopy instrument provides numerous advantages over the similar instruments of the prior art. The snare 20 can easily surround the object and be closed gradually to the desired configuration for grasping the object. The basket portion 15 as formed by the basket wires 30 holds the object and prevents the object from falling out of the grasp of the snare 20. The hooks 45, where applicable, aid in grasping the object without risking removal thereof from the snare 20 and/or basket wires 30, thereby allowing the object to be retrieved while remaining in one piece. Finally, the optional net 60 prevents any smaller or broken away portions of the object from falling out of or through the snare 20 and/or basket wires 30. The resulting endoscopy instrument 10 is accordingly more maneuverable, can be gradually opened or collapsed to accommodate variable sizes and configurations of the object, and can be positioned in any of a variety of different orientations for approaching and grasping the object in a variable manner.

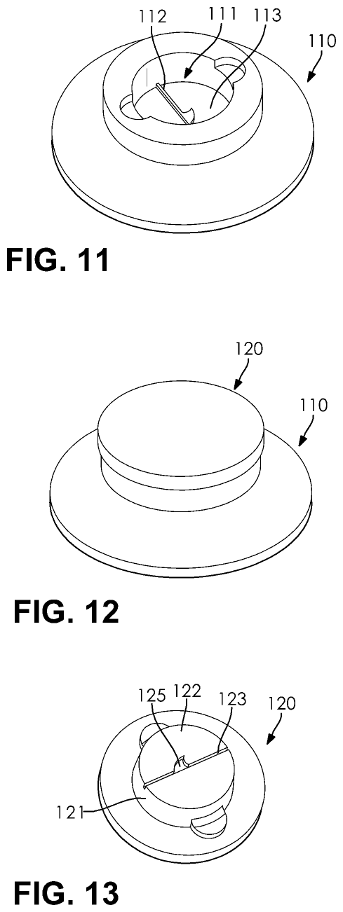

Referring now to FIGS. 11-16, a method of forming one of the barbs 45 relative to a corresponding one of the basket wires 30 is disclosed. The disclosed method allows for such barbs 45 to be manufactured quickly and easily with respect to an endoscopy instrument 10 of substantially any dimensions, and allows for each of the resulting barbs 45 to have substantially any desired shape and configuration suitable for application to one of the basket wires 30.

As shown in FIG. 11, the method includes an initial step of forming a first mold 110 having a cavity 111 and a projection 112. The first mold 110 may alternatively be referred to as a preliminary mold 110 herein. The cavity 111 forms an opening within the first mold 110 for receiving a molding material therein. The projection 112 projects outwardly from a base surface 113 defining a planar surface of the cavity 111. The projection 112 has the general configuration of one of the segments 33, 34, 35 having one of the barbs 45 projecting laterally therefrom. Specifically, the projection 112 resembles a ridge or rib having a lateral projection extending therefrom.

The first mold 110 may be formed by an additive printing process (3-D printing process) to allow the first mold 110 to be quickly and easily produced to account for substantially any desired configuration of the resulting endoscopy instrument 10, including instruments 10 having different dimensions or different applications. For example, it should be readily apparent that the first mold 110 may be quickly and easily produced to include multiple projections 112 corresponding to multiple basket wires 30 or a single projection 112 having multiple barb-like shapes formed therein for forming multiple spaced apart barbs 45 on a single basket wire 30, as desired. A shape and configuration of each of the barbs 45 may also be easily modified by adjusting the perimeter shape of the disclosed projection 112, as described hereinafter. The first mold 110 may be formed from a substantially rigid first material.

As shown in FIG. 12, the method then includes a step of forming a second mold 120 within the cavity 111 of the first mold 110. Specifically, a second material is received into the cavity 111 to cause the second mold 120 to have an exposed portion having a shape and configuration that is inverted relative to that of the cavity 111, which includes a shape that is inverted relative to the projection 112 formed within the cavity 111. The second material may be an elastomeric material, such as a 2-part silicone rubber. The step of forming the second mold 120 may include a step of vibrating the first mold 110 and the second material received within the cavity 111 in order to promote the migration of air bubbles to an exposed surface of the second material, thereby ensuring a quality of the second mold 120. The second mold 120 may alternatively be referred to as the barb-forming mold 120 herein.

Once appropriately cured or otherwise solidified, the second mold 120 may be removed from the first mold 110 to result in the second mold 120 having the configuration shown in FIG. 13. Specifically, the second mold 120 includes a body 121 having a planar surface 122 and a channel 123 formed as an indentation into the planar surface 122 of the body 121. The channel 123 extends from one side (edge) of the body 121 to an opposing side (edge) thereof, which corresponds to diametrically opposing sides of a cylindrical body 121 with reference to the embodiment illustrated in FIG. 13. A barb recess 125 projects laterally from the channel 123 and is similarly provided as an indentation relative to the planar surface 122. The barb recess 125 includes a base surface 126, a first side surface 127, and a second side surface 128. The base surface 126 is spaced axially from the planar surface 122, and the side surfaces 127, 128 extend axially to join the base surface 126 to the planar surface 122 to one side of the channel 123. As explained previously, the side surfaces 127, 128 may include a combination of planar surfaces, concave surfaces, and/or convex surfaces for forming a desired shape of the resulting barb 45. In the present embodiment, the first side surface 127 is concave in shape while the second side surface 128 is convex in shape, thereby resulting in a hook or shark-fin shaped barb 45.

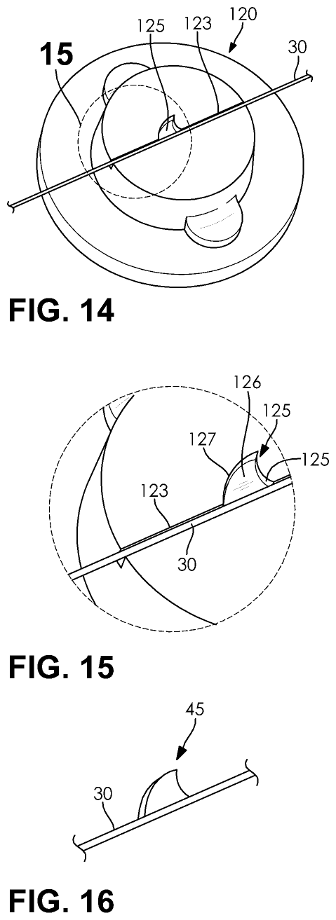

As shown in FIGS. 14 and 15, the method then includes a step of positioning an elongate element into the channel 123 of the second mold 120 for forming one of the barbs 45 at the position of the barb recess 125. As shown, the elongate element may be representative of one of the segments 33, 34, 35 of one of the described basket wires 30, but the elongate element may alternatively be representative of one of the snare segments 23, 24, as desired. Once so positioned, the method includes a step of inserting a third material into the barb recess 125 of the second mold 120, wherein the third material may be a UV curable adhesive. The inserting step may include the third material being added in droplet form to the recess 125, as desired. However, any process suitable for delivering the third material to the recess 125 may be utilized without necessarily departing from the scope of the present invention.

The method then includes a step of curing the third material to form the barb 45 at the desired position on the basket wire 30 (or snare segment 23, 24). The curing may include the third material being exposed to UV light to cure the UV curable adhesive. The curing of the third material may include at least some of the third material curing into a shape at least partially surrounding the basket wire 30, thereby establishing the connection of the barb 45 to the basket wire 30. The basket wire 30 having the barb 45 formed thereon is then removed from the second mold 120 to finalize the formation process thereof. The completed barb 45 is shown as extending laterally from the basket wire 30 in FIG. 16.

The described process beneficially allows for the barbs 45 to be formed to have substantially any shape and configuration, as well as any desired placement and number, along the wire received within the channel 123. The process can easily be adjusted or customized due to the use of the additive printing process is forming the preliminary mold 110, which in turn is quickly and easily utilized in forming the barb-forming mold 120. The disclosed method accordingly allows for the creation of new and potentially customized endoscopy instruments 10 via a simple and repeatable process.

From the foregoing description, one ordinarily skilled in the art can easily ascertain the essential characteristics of this invention and, without departing from the spirit and scope thereof, can make various changes and modifications to the invention to adapt it to various usages and conditions.

What is claimed is:

1. An endoscopy instrument configured to be adjustable between a retracted position and an extended position, the endoscopy instrument comprising:
a snare forming a loop extending from a proximate end to a distal end; and
a plurality of basket wires, each of the basket wires including a proximate end coupled to the proximate end of the snare and a distal end coupled to the distal end of the snare, the snare and the plurality of the basket wires cooperating to form a basket portion configured to contract when the endoscopy instrument is adjusted from the extended position towards the retracted position.

2. The endoscopy instrument of claim 1, wherein each of the basket wires is concave towards the snare when the endoscopy instrument is in the extended position.

3. The endoscopy instrument of claim 1, wherein the basket portion is dome shaped.

4. The endoscopy instrument of claim 3, wherein the basket portion is one of hemi-ellipsoidal dome shaped or semi-ellipsoidal dome shaped.

5. The endoscopy instrument of claim 1, wherein the basket portion is elongated in one direction when the endoscopy instrument is in the extended position.

6. The endoscopy instrument of claim 1, further comprising an elongate stem, the proximate end of the snare coupled to an end of the stem.

6. oscopy instrument of claim 6, further comprising a tubular member having a passageway formed therethrough, wherein the stem is slidably disposed within the passageway.

8. The endoscopy instrument of claim 7, wherein the endoscopy instrument is adjusted from the extended position to the retracted position when the basket portion enters the passageway during a sliding of the stem within the passageway.

9. The endoscopy instrument of claim 1, wherein the snare is elliptical in shape when the endoscopy instrument is in the extended position.

10. The endoscopy instrument of claim 1, wherein at least one of the basket wires includes a barb projecting therefrom.

11. The endoscopy instrument of claim 10, wherein the barb projects towards a plane defined by the snare when the endoscopy instrument is in the extended position.

12. The endoscopy instrument of claim 10, wherein the barb is formed from a UV curable material molded onto the at least one of the basket wires.

13. The endoscopy instrument of claim 1, further comprising a net coupled to the basket portion.

14. The endoscopy instrument of claim 1, wherein each of the plurality of the basket wires is angularly offset from the snare and an adjacent one of the plurality of the basket wires when the endoscopy instrument is in the extended position.

15. The endoscopy instrument of claim 1, wherein a width and a height of the basket portion are each reduced as the endoscopy instrument is adjusted from the extended position to the retracted position.

16. A method of manufacturing an endoscopy instrument including the steps of:
providing a barb-forming mold having a channel and a barb recess formed therein, the barb recess extending laterally from the channel;
positioning an elongate element of the endoscopy instrument within the channel of the barb-forming mold;
inserting a curable material into the barb recess; and
curing the curable material to form a barb coupled to the elongate element.

17. The method of claim 16, wherein the curable material is a UV curable adhesive and the curing includes exposing the curable material to UV light.

18. The method of claim 16, further including a step of forming a preliminary mold using an additive printing process, the barb-forming mold formed during a molding process utilizing the preliminary mold.

19. The method of claim 18, wherein the molding process utilizing the preliminary mold includes vibrating the preliminary mold.

20. The method of claim 18, wherein the preliminary mold is formed from a rigid material and the barb-forming mold is formed from an elastomeric material.
